(19) United States
(12) Patent Application Publication
(10) Pub. No.: US 2025/0262612 A1
(43) Pub. Date: Aug. 21, 2025

(54) SOLID ACID ALKYLATION CATALYST, AND PREPARATION METHOD THEREFOR AND USE THEREOF

(71) Applicant: PetroChina Company Limited, Beijing (CN)

(72) Inventors: Yan Cui, Beijing (CN); Shiyong Xing, Beijing (CN); Minghan Han, Beijing (CN); Xiaohua Wang, Beijing (CN); Chengyu Guo, Beijing (CN); Yin Xie, Beijing (CN); Hongyue Yu, Beijing (CN); Liang Qiao, Beijing (CN); Kebin Chi, Beijing (CN); Shang Zhang, Beijing (CN)

(21) Appl. No.: 18/857,283

(22) PCT Filed: Dec. 13, 2022
(86) PCT No.: PCT/CN2022/138664

§ 371 Date: Oct. 16, 2024

(30) Foreign Application Priority Data
Nov. 8, 2022 (CN) 202211390303.X

(51) Int. Cl. B01J 29/70; B01J 37/10; C07C 2/66

(52) U.S. Cl. CPC B01J 29/7038; B01J 37/10; C07C 2/66; C07C 2529/70

(57) ABSTRACT

A solid acid alkylation catalyst, and a preparation method therefor and a use thereof. The preparation method comprises the following steps: S1, mixing an aluminum source, water, an alkali source, a template agent, a silicon source and a seed crystal to form gel, and carrying out a crystallization reaction to obtain a molecular sieve having an MWW structure, wherein the template agent is composed of a main template agent and an auxiliary template agent in a molar ratio of 0.5-20:1; S2, roasting the molecular sieve having the MWW structure obtained in S1 to remove the template agent, then carrying out ammonium exchange, and roasting again to obtain an H-type molecular sieve; and S3, mixing the H-type molecular sieve obtained in S2 with an inorganic oxide and a nitric acid solution, and performing kneading and shaping to obtain the solid acid alkylation catalyst. By using the method for synergistically assisting crystallization by a seed crystal and two template agents, a molecular sieve having an MWW structure is directly synthesized without using hexamethyleneimine, piperidine and homopiperazine, and the solid acid alkylation catalyst is further obtained.

### FIELD OF TECHNOLOGY

The present disclosure relates to a solid acid catalyst, and specifically to a solid acid alkylation catalyst and the preparation method and use thereof.

### BACKGROUND OF ART

Alkylation is a very important type of reaction in petrochemical industry, and the alkylation reaction of aromatic hydrocarbons with olefins to produce alkyl-substituted aromatic hydrocarbons is one of the most important series. Common catalysts can be divided into L-acid catalysts and B-acid catalysts. L-acid catalysts, mainly represented by anhydrous AlCl₃, have the advantages of low price, good catalytic activity and mature technology. However, the production process is accompanied by the generation of a large amount of aluminum-containing waste liquid and too many side reactions, so that such catalysts have been substantially eliminated from the market. B-acid catalysts, mainly represented by HF, H₂SO₄ and H₃PO₄, and the use of liquid acid is mainly concentrated in the alkylation reaction of long chain olefins (carbon number>8), because the reaction of the long chain olefins can easily lead to the deactivation of the catalysts due to the accumulation of carbon. However, due to the strong corrosiveness of HF and H₂SO₄, the requirements on the material of the equipment are very high, and the production process will also be accompanied by the emission of various types of wastes, and the environmental protection cost is very high. As a result, the development of solid acid catalysts is considerably necessary.

Macroporous 12-membered ring molecular sieves have strong internal channel diffusion performance, which has an outstanding effect for alleviating the rapid deactivation of catalysts caused by channel blockage. However, traditional 12-membered ring macroporous molecular sieves, such as octahedral zeolite and beta zeolite, are prone to be deactivated due to blockage of the internal channels of the crystals by macromolecules. Although different kinds of 12-membered ring macroporous molecular sieves are used as the main active components of the catalysts in the prior art, and the diffusion performance of the molecular sieves is further improved by modification of the molecular sieves, there are still the problems such as cumbersome costs for production, presence of strong corrosive halogens, and decrease in the stability of the molecular sieves.

The MWW-structured molecular sieve has a very typical two-dimensional lamellar morphology, and the lamellar structure thereof consists of several basic monolayers stacked together, which also forms a unique channel structure, mainly including two-dimensional sinusoidal 10-membered ring channels in the plane of ab-axis and 12-membered ring supercage and half-supercage structures in the direction of c-axis. The 10-membered ring channel have an opening size of 0.41 nm×0.51 nm and are distributed outside the supercage structure; the 12-membered ring supercage having a size of 0.71 nm×0.71 nm×1.82 nm is connected to six identical supercages in the surrounding ab-axis plane through 10-membered ring; the half-supercages distributed on the surface of the lamellae have a size of 0.71 nm×0.71 nm×0.8 nm, and 12-membered ring openings thereof are very favorable for the diffusion of the reactants with excellent catalytic reaction performance for macromolecular reactions, and have been widely used in reaction processes such as alkylation.

The MWW-structured molecular sieve can be subdivided into many varieties according to the difference in stacking of the basic monolayers, mainly including MCM-22 (P) molecular sieve with hydrogen bonding between monolayers. MCM-49 molecular sieve having oxygen bridge bond between monolayers. MCM-36 molecular sieve having a pillared structure between layers. MCM-56 molecular sieve having disordered interlayer stacking, ITQ-2 molecular sieve having a single-layer structure, and the like. Among them, the MCM-22 molecular sieve is special, which can be prepared into MCM-22 molecular sieve (having the same crystal structure as that of MCM-49 molecular sieve) after dehydration of interlayer silica hydroxyl group by roasting to form oxygen bridge bond, into MCM-36 molecular sieve after swelling and pillaring, or into ITQ-2 molecular sieve after swelling and delamination. Therefore, the MCM-22 (P) molecular sieve has a good potential for modification.

The template agents used for direct induction synthesis of the MWW-structured molecular sieve generally include hexamethyleneimine, piperidine, and homopiperazine. Among them, hexamethyleneimine is the most widely used, but it has the disadvantages of volatility, flammability and high toxicity. Piperidine is a precursor chemical and difficult to obtain. Homopiperazine is expensive and difficult to apply. Therefore, it is very important to develop an inexpensive and green synthesis method for the MWW-structured molecular sieve.

[Synthesis and application study of MCM-22 molecular sieves [D]. Dalian. Dalian Institute of Chemical Physics. Chinese Academy of Sciences. 2006.] achieves the synthesis of MCM-22 molecular sieve using hexamethyleneimine and cyclohexylamine as double template agents, which effectively reduces the amount of hexamethyleneimine.

CN02145233.4 discloses a method of synthesizing MCM-22 molecular sieve using pentamethyleneimine, hexamethyleneimine or heptamethyleneimine as a template agent and recycling the mother liquor, which effectively reduces the amount of template agent. CN201310538868.2 discloses a method of synthesizing MCM-22 molecular sieve using hexamethyleneimine and cyclohexylamine as double template agents. CN201110216909.7 discloses a method for synthesizing MCM-22 molecular sieve by using an acidified hexamethyleneimine templating agent and refluxing. [Microporous and Mesoporous Materials. 2019, 276:192-200.] realizes the synthesis of MCM-49 molecular sieve by co-induction with a crystal seed and cyclohexylamine. It can be found that the synthesis cannot be realized directly with crystal seeds due to the specificity of the MWW-structured molecular sieve. By adding cyclohexylamine on the basis of the crystal seed, the 10-membered ring channels can be supported to construct a complete MCM-49 molecular sieve, but the hydrogen bonding spacing between the basic layers cannot be realized. So far, there is no precedent for the synthesis of MCM-22 molecular sieves without using hexamethyleneimine, piperidine, or homopiperazine, and thus the preparation of MCM-22 is bound to have problems brought about by template agents.

### SUMMARY OF THE INVENTION

In order to solve the above problems, an object of the present disclosure is to provide a solid acid alkylation catalyst and the preparation method and use thereof. By synergetically assisting a crystallization reaction with a crystal seed and two template agents, a MWW-structured molecular sieve is directly synthesized without hexamethyleneimine, piperidine, or homopiperazine, thereby producing the solid acid alkylation catalyst. In order to achieve the above object, the present disclosure provides a method for preparing a solid acid alkylation catalyst, which comprises the following steps:
S1: mixing an aluminum source, water, an alkali source, a template agent, a silicon source, and a crystal seed to form a gel, and subjecting the gel to a crystallization reaction to obtain a MWW-structured molecular sieve; wherein the template agent is composed of a primary template agent and a secondary template agent in a molar ratio of 0.5-20:1; the primary template agent is cyclohexylamine, and the secondary template agent comprises at least one of diisopropylamine, di-n-butylamine, diisobuty lamine, 1,4-diazabicyclo[2.2.2]octane, 1,6-hexanediamine and N,N,N,N-tetramethyl-1,6-hexanediamine (CAS No.: 111-18-2, molecular formula: C₁₀H₂₄N₂);
S2: roasting the MWW-structured molecular sieve obtained from S1 to remove the template agent, followed by ammonium exchange and roasting again, to obtain an H-type molecular sieve: and
S3: mixing the H-type molecular sieve obtained from S2 with an inorganic oxide and an inorganic acid solution, kneading and shaping, drying and then roasting, to obtain the solid acid alkylation catalyst.

In the preparation method of the present disclosure, the primary template agent and the secondary template agent are both low-toxic and inexpensive organic amines. Among them, the primary template agent can cooperate with the crystal seed to realize the construction of the basic layer structure of the molecular sieve: and the secondary template agent adopts an aliphatic amine with a sp³ hybridized N atom, and it can form interlayer hydrogen bonding with Si—OH on the surface of the monolayer structure by means of holes in a free orbital of the sp³ hybridized orbitals of the nitrogen atom, and the process of forming the hydrogen bonding does not affect the overall crystallization effect of the molecular sieve. Thus, in some embodiments, the MWW-structured molecular sieve obtained by the preparation method described above has interlayer hydrogen bonding formed of Si—OH and the secondary template agent.

The preparation method of the present disclosure allows for the synthesis of a MWW-structured molecular sieve having interlayer hydrogen bonding without hexamethyleneimine, piperidine, or homopiperazine by synergetic interaction between the primary template agent, the secondary template agent, and the crystal seed.

In the method for preparing a solid acid alkylation catalyst, preferably, in the gel in SI, the molar ratio of the silicon source in terms of SiO₂, the aluminum source in terms of Al₂O³, the alkali source in terms of alkali metal oxide and template agent is 1:0.005-0.05:0.03-0.50:0.1-0.75.

The mass ratio of the crystal seed on dry mass basis to the silicon source in terms of SiO₂ is 0.01-0.25:1.

In the method for preparing a solid acid alkylation catalyst, preferably, in S1, the crystal seed is a molecular sieve having a MWW topology.

In the method for preparing a solid acid alkylation catalyst, preferably, in S1, the crystal seed comprises a MCM-22 molecular sieve or a MCM-49 molecular sieve without template agent removal. The crystal seed is preferably a MCM-22 molecular sieve without template agent removal, which has interlayer hydrogen bonding, and is favorable for obtaining a MWW-structured molecular sieve having interlayer hydrogen bonding and a structure similar to that of the MCM-22 molecular sieve, when used as the crystal seed in the above preparation process.

In the method for preparing a solid acid alkylation catalyst, preferably, in S1,the silicon source comprises at least one of silica sol, solid silica gel, white carbon black and water glass.

In the method for preparing a solid acid alkylation catalyst, preferably, in S1, the alkali source comprises sodium hydroxide and/or potassium hydroxide.

In the method for preparing a solid acid alkylation catalyst, preferably, in S1, the aluminum source comprises at least one of sodium metaaluminate, aluminum sulfate, alumina and pseudo boehmite.

In the method for preparing a solid acid alkylation catalyst, preferably, in S1, the crystallization reaction temperature is 120-170° C., and the reaction time is 12-120 h.

In the method for preparing a solid acid alkylation catalyst, preferably, the step S1 specifically comprises mixing the aluminum source, water, the alkali source, the primary template agent, and the secondary template agent to obtain an intermediate solution, slowly adding the silicon source to the intermediate solution and mixing well, adding the crystal seed, mixing them to form a gel, and subjecting the gel to a crystallization reaction to obtain the MWW-structured molecular sieve. The synthesis in the above order facilitates uniform dispersion of each raw material and promotes the crystallization reaction. The primary template agent and the secondary template agent are added after the aluminum source, which facilitates the aluminum source to be completely dissolved and uniformly dispersed in the solution. The crystal seed is added after the slow addition of the silicon source, which can improve the homogeneity and stability of the reaction system, and avoid the problem of forming a colloid with the aluminum source after rapid addition of the silicon source to cause a non-uniform dispersion of the reaction system.

The MWW-structured molecular sieve obtained from S1 of the present disclosure has interlayer hydrogen bonding and a structure similar to that of a MCM-22 molecular sieve which has not been roasted (i.e., without template agent removal), and thus it can be regarded as a precursor of the MCM-22 molecular sieve (which can be labeled as a MCM-22 (P) molecular sieve). The MWW-structured molecular sieve of the present disclosure has good modification potential, for example, it can be prepared into a MCM-36 molecular sieve after swelling and pillaring, or into an ITQ-2 molecular sieve after swelling and delamination. It has been found that in the present disclosure, the MCM-49 molecular sieve has no interlayer hydrogen bonding and could not be swelled and delaminated; the MCM-22 molecular sieve produced with a conventional template agent (hexamethyleneimine) and the MWW-structured molecular sieve provided by the present disclosure have the ability to swell and delaminate because both of them have interlayer hydrogen bonding. Compared to the MCM-22 molecular sieve produced with a conventional template agent (hexamethylenimine), the MWW-structured molecular sieve provided by the present disclosure has less difficulty in delamination, which can be reflected in the fact that the pH of the alkaline environment required for delamination is closer to neutral. This indicates that the interlayer hydrogen bonding strength of the MWW-structured molecular sieve provided by the present disclosure is more moderate than that of the MCM-22 molecular sieve, which is more conducive to retaining the integrity of the lamellar crystal structure of the molecular sieve, improving the stability of the molecular sieve, and decreasing the loss of the catalytically active sites when the molecular sieve is modified by pillaring or delamination. Therefore, it is conducive to improving the catalytic activity and stability of the molecular sieve when it is applied in alkylation reaction, isomerization reaction or cracking reaction.

In the method for preparing a solid acid alkylation catalyst, preferably, in S2, the roasting is each carried out under an air atmosphere, the roasting temperature is each independently 490-590° C., and the roasting time is each independently 0.5-72 h.

In the method for preparing a solid acid alkylation catalyst, preferably, in S2, the ammonium exchange is carried out in an ammonium nitrate solution with a concentration of 0.1-10 mol/L; the ammonium exchange temperature is 30-120° C., and the ammonium exchange time is 0.5-72 h.

In the method for preparing a solid acid alkylation catalyst. preferably, in S3, the mass ratio of the H-type molecular sieve to the inorganic oxide is 10-95:5-90.

In the method for preparing a solid acid alkylation catalyst, preferably, in S3, the inorganic oxide comprises at least one of aluminium oxide, pseudo boehmite, boehmite and aluminum hydroxide.

In the method for preparing a solid acid alkylation catalyst, preferably, in S3, the inorganic acid solution comprises at least one of nitric acid solution, phosphoric acid solution, hydrochloric acid, sulfuric acid solution. In the present disclosure, the inorganic acid solution is used to emulsify the inorganic oxide to form a binder.

In the method for preparing a solid acid alkylation catalyst, preferably, in S3, the roasting is carried out under an air atmosphere, the roasting temperature is 490-590° C. and the roasting time is 0.5-72 h.

The present disclosure further provides a solid acid alkylation catalyst obtained from the method for preparing a solid acid alkylation catalyst.

The present disclosure further provides use of the solid acid alkylation catalyst obtained from the method for preparing a solid acid alkylation catalyst in the alkylation reaction of an aromatic hydrocarbon with an olefin.

In the use, preferably, the aromatic hydrocarbon is selected from at least one of benzene, alkylbenzene, naphthalene, alkylnaphthalene, anthracene and alkylanthracene, and more preferably at least one of benzene, alkylbenzene, naphthalene and anthracene.

In the use, preferably, the olefin is selected from olefins having 2 to 20 carbon atoms, more preferably olefins having 2 to 15 carbon atoms.

In the use, preferably, the alkylation reaction conditions include a reaction temperature of 100-385°° C., a reaction pressure of 0.5-7 MPa, and a weight hourly space velocity of 0.1-12 h⁻¹; more preferably, the alkylation reaction conditions include a reaction temperature of 120-350° C., a reaction pressure of 0.5-5 MPa, and a weight hourly space velocity of 0.5-10 h⁻¹.

In the use, preferably, the feeding molar ratio of the aromatic hydrocarbon to the olefin is 0.05-200.

The technical solution provided by the present disclosure has the following beneficial effects:
(1) In the solid acid alkylation catalyst provided by the present disclosure, the catalytically active component, an H-type molecular sieve, is made of the MWW-structured molecular sieve by ammonium exchange, wherein the MWW-structured molecular sieve realizes the construction of the basic layer structure through the crystal seed and the primary template agent, realizes the construction of the interlayer hydrogen bonding through the secondary template agent, and has the characteristics similar to the MCM-22 (P) molecular sieve.
(2) The primary template agent and the secondary template agent used in the present disclosure are low-toxic and readily available organic amines with low cost, and highly toxic precursor chemical template agents such as hexamethyleneimine, piperidine, and homopiperazine are not used. This reduces the contamination and cost of the method for preparing a solid acid alkylation catalyst. In addition, the prepared solid acid alkylation catalyst has high alkylation catalytic activity and product selectivity, which can greatly improve the operability of the production of MWW-structured molecular sieve, and have strong practical application significance.

### BRIEF DESCRIPTION OF THE DRAWINGS

FIG. 1 shows an XRD spectrum of the crystal seed;

FIG. 2 shows a SEM image of the crystal seed;

FIG. 3 shows an XRD spectrum of the MWW-structured molecular sieve synthesized in Example 1;

FIG. 4 shows a SEM image of the MWW-structured molecular sieve synthesized in Example 1;

FIG. 5 shows an XRD spectrum of the MWW-structured molecular sieve synthesized in Example 2;

FIG. 6 shows a SEM image of the MWW-structured molecular sieve synthesized in Example 2;

FIG. 7 shows an XRD spectrum of the molecular sieve synthesized in Comparative Example 1;

FIG. 8 shows a SEM image of the molecular sieve synthesized in Comparative Example 1;

FIG. 9 shows an XRD spectrum of the molecular sieve synthesized in Comparative Example 3; and

FIG. 10 shows a SEM image of the molecular sieve synthesized in Comparative Example 3.

### DETAILED DESCRIPTION OF PREFERRED EMBODIMENTS

In order to have a clearer understanding of the technical features, objects and beneficial effects of the present disclosure, the technical solutions of the present disclosure will now be described in details below, but it is not to be construed as a limitation of the implementable scope of the present disclosure.

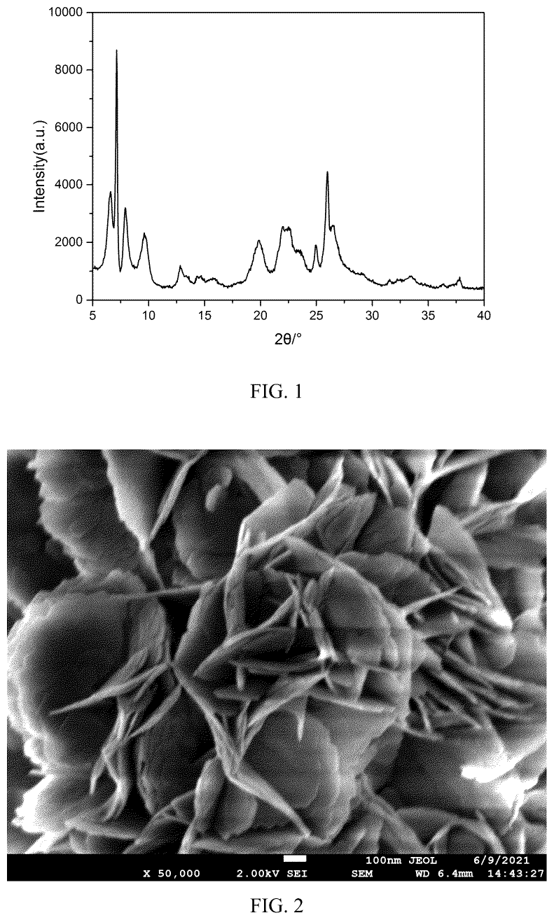

The raw materials used in the examples of the present disclosure are as follows:
Silicon source: silica sol (SiO₂ content of 40%), solid silica gel (SiO₂ content of 95%), white carbon black (SiO₂ content of 93%), ethyl orthosilicate (98% purity);
Aluminum source: sodium metaaluminate (Al₂O₃ content of 41%), aluminum sulfate (Al₂O₃ content of 15%), alumina (Al₂O₃ content of 95%), pseudo boehmite (Al₂O₃ content of 70%);
Alkali source: sodium hydroxide (99% purity), potassium hydroxide (99% purity); Primary template agent: cyclohexylamine (99% purity);
Secondary template agent: diisopropylamine (99% purity), di-n-butylamine (99% purity), diisobutylamine (99% purity), 1,4-diazabicyclo[2.2.2]octane (99% purity), 1,6-hexanediamine (99% purity), N,N,N,N-tetramethyl-1,6-hexanediamine (99% purity);
Crystal seed: MCM-22 molecular sieve from Mobil, without template agent removal by roasting; after characterization, its XRD spectrum and SEM image are shown in FIGS. 1 and 2, respectively.
Other: deionized water.

The present disclosure would be described below in connection with specific examples and figures.

### Example 1

This example provides a MWW-structured molecular sieve, prepared as follows:
1.87 g of sodium hydroxide was added to 120 g of deionized water and dissolved with stirring; 2.15 g of sodium metaaluminate was added and dissolved with continuous strong stirring for 1 h; 9.50 g of cyclohexylamine was added slowly with continuous strong stirring for 0.5 h; 6.50 g of diisopropylamine was added slowly with continuous strong stirring for 0.5 h; 80 g of silica sol was added slowly with continuous strong stirring for 3 h; a crystal seed was added in an amount of 3% of the effective SiO₂ mass in the silica sol with continuous strong stirring for 1 h, to produce a crystallized gel; the crystallized gel was crystallized at 140° C. for 60 h; at the end of crystallization, the temperature was reduced to room temperature; the product was washed with deionized water, filtered and dried at 120° C. for 4 h, to obtain the MWW-structured molecular sieve.

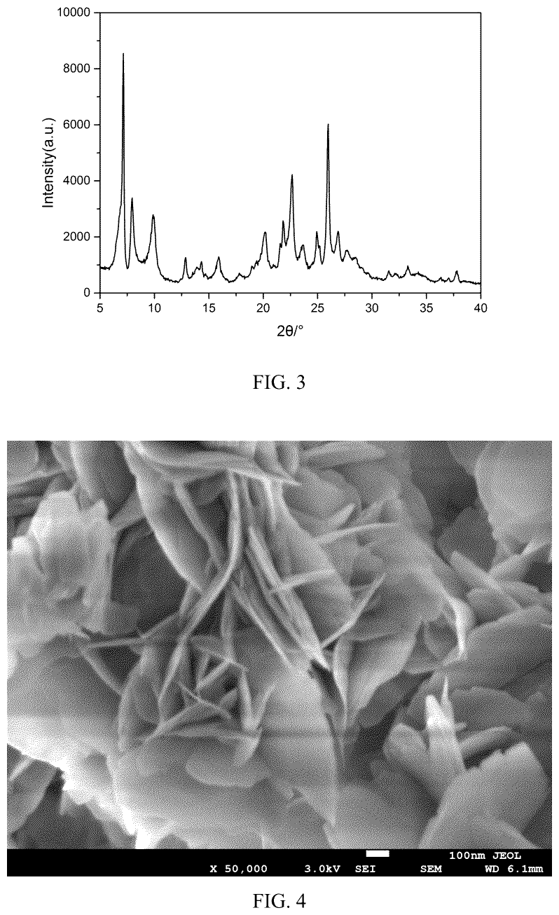

The MWW-structured molecular sieve obtained in this example was characterized by X-ray diffraction (XRD) and scanning electron microscopy (SEM) analysis. The XRD spectrum and SEM image are shown in FIGS. 3 and 4, respectively, which indicate that the molecular sieve has typical characteristic peaks of MWW structure, and the morphology of the molecular sieve product is in the form of flower clusters of stacked nanosheets.

The MWW-structured molecular sieve obtained in this example was characterized by specific surface area and pore size (BET) analysis after roasting at 540° C. in air atmosphere, and the results of BET test are shown in Table 1. In Table 1, S_{BET} is the specific surface area, S_{mic} is the micropore surface area, Sₑₓₜ is the mesopore surface area, Vₚₒᵣₑ is the pore volume, and V_{mic} is the micropore volume.

### Example 2

This example provides a MWW-structured molecular sieve, prepared as follows:
1.87 g of sodium hydroxide was added to 120 g of deionized water and dissolved with stirring; 2.50 g of sodium metaaluminate was added and dissolved with continuous strong stirring for 1 h; 9.00 g of cyclohexylamine was added slowly with continuous strong stirring for 0.5 h; 8.00 g of 1,6-hexanediamine was added slowly with continuous strong stirring for 0.5 h; 80 g of silica sol was added slowly with continuous strong stirring for 3 h; a crystal seed was added in an amount of 5% of the effective SiO₂ mass in the silica sol with continuous strong stirring for 1 h, to produce a crystallized gel; the crystallized gel was crystallized at 145° C. for 72 h; at the end of crystallization, the temperature was reduced to room temperature; the product was washed with deionized water, filtered and dried at 120° C. for 4 h, to obtain the MWW-structured molecular sieve.

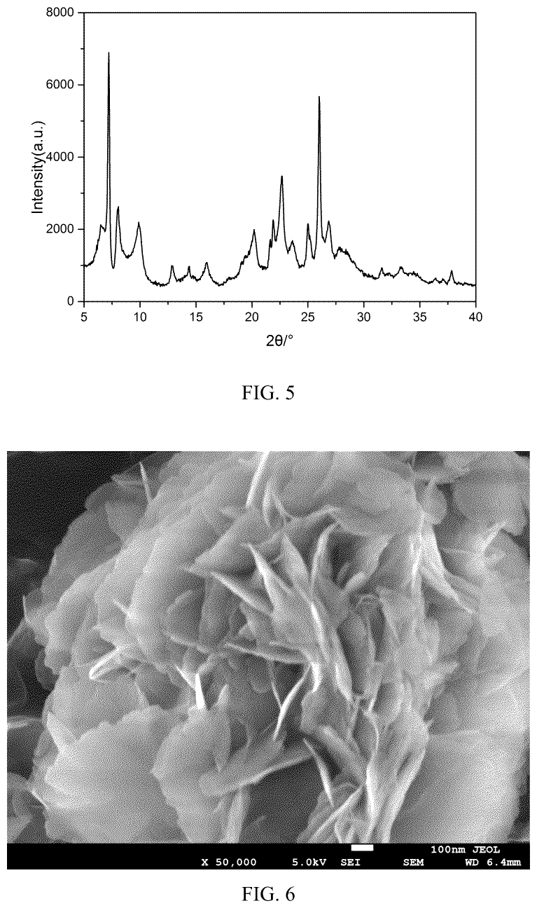

The MWW-structured molecular sieve obtained in this example was characterized by XRD and SEM analysis. The XRD spectrum and SEM image are shown in FIGS. 5 and 6, respectively, which indicate that the molecular sieve has typical characteristic peaks of MWW structure, and the morphology of the molecular sieve product is in the form of flower clusters of stacked nanosheets.

The MWW-structured molecular sieve obtained in this example was characterized by BET analysis after roasting at 540° C. in air atmosphere, and the results of BET test are shown in Table 1.

### Example 3

This example provides a MWW-structured molecular sieve, prepared as follows:
1.95 g of sodium hydroxide was added to 130 g of deionized water and dissolved with stirring; 2.33 g of sodium metaaluminate was added and dissolved with continuous strong stirring for 1 h; 9.00 g of cyclohexylamine was added slowly with continuous strong stirring for 0.5 h; 9.00 g of 1,4-diazabicyclo[2.2.2]octane was added slowly with continuous strong stirring for 0.5 h; 80 g of silica sol was added slowly with continuous strong stirring for 3 h; a crystal seed was added in an amount of 5% of the effective SiO₂ mass in the silica sol with continuous strong stirring for 1 h, to produce a crystallized gel; the crystallized gel was crystallized at 140° C. for 72 h; at the end of crystallization, the temperature was reduced to room temperature: the product was washed with deionized water, filtered and dried naturally for 48 h, to obtain the MWW-structured molecular sieve.

The product obtained in this example was characterized by XRD and SEM analysis and characterized by BET analysis (after roasting at 540° C. in air atmosphere). It can be seen that it has typical characteristic peaks of MWW structure, and the morphology of the molecular sieve product is in the form of flower clusters of stacked nanosheets. The results of BET test are shown in Table 1.

### Example 4

This example provides a MWW-structured molecular sieve, prepared as follows:
1.93 g of sodium hydroxide was added to 150 g of deionized water and dissolved with stirring; 1.50 g of sodium metaaluminate was added and dissolved with continuous strong stirring for 1 h; 12.25 g of cyclohexylamine was added slowly with continuous strong stirring for 0.5 h; 8.50 g of N,N,N,N-tetramethyl-1,6-hexanediamine was added slowly with continuous strong stirring for 0.5 h; 80 g of silica sol was added slowly with continuous strong stirring for 3 h; a crystal seed was added in an amount of 10% of the effective SiO₂ mass in the silica sol with continuous strong stirring for 1 h, to produce a crystallized gel; the crystallized gel was crystallized at 140° C. for 100 h; at the end of crystallization, the temperature was reduced to room temperature; the product was washed with deionized water, filtered and dried at 120° C. for 4 h, to obtain the MWW-structured molecular sieve.

The product obtained in this example was characterized by XRD, SEM and BET analysis (after roasting at 540° C. in air atmosphere). It can be seen that it has typical characteristic peaks of MWW structure, and the morphology of the molecular sieve product is in the form of flower clusters of stacked nanosheets.

### Example 5

This example provides a MWW-structured molecular sieve, prepared as follows:
2.21 g of sodium hydroxide was added to 175 g of deionized water and dissolved with stirring; 1.25 g of sodium metaaluminate was added and dissolved with continuous strong stirring for 1 h; 7.55 g of cyclohexylamine was added slowly with continuous strong stirring for 0.5 h; 10.50 g of diisobutylamine was added slowly with continuous strong stirring for 0.5 h; 80 g of silica sol was added slowly with continuous strong stirring for 3 h; a crystal seed was added in an amount of 15% of the effective SiO₂ mass in the silica sol with continuous strong stirring for 1 h, to produce a crystallized gel; the crystallized gel was crystallized at 140° C. for 120 h; at the end of crystallization, the temperature was reduced to room temperature: the product was washed with deionized water, filtered and dried naturally for 48 h, to obtain the MWW-structured molecular sieve.

The product obtained in this example was characterized by XRD and SEM analysis and characterized by BET analysis (after roasting at 540° C. in air atmosphere). It can be seen that it has typical characteristic peaks of MWW structure, and the morphology of the molecular sieve product is in the form of flower clusters of stacked nanosheets. The results of BET test are shown in Table 1.

### Example 6

This example provides a solid acid alkylation catalyst, prepared as follows:
the molecular sieve product of Example 1 was taken and roasted at 540° C. under air atmosphere for 5 h to remove the template agent; it was then subjected to ammonium exchange in 1 mol/L ammonium nitrate solution at 80° C. for 2 h and roasted again at 540° C. for 4 h under air atmosphere to obtain an H-type molecular sieve;
90 g of the H-type molecular sieve was mixed evenly with 15 g of pseudo boehmite, and 55 g of nitric acid solution was added gradually while kneading, and extruded into a φ2.0 mm cylindrical catalyst, which was then truncated into a cylindrical catalyst with a length of 2.5 mm: the catalyst was dried at room temperature for 24 h, and then roasted at 550° C. for 6 h to obtain a finished solid acid alkylation catalyst.

The solid acid alkylation catalyst obtained in this example was applied to catalyze the alkylation reaction of an aromatic hydrocarbon with an olefin as follows:
2 g of the finished solid acid alkylation catalyst was loaded into a fixed-bed reactor and a mixture of benzene and ethylene was introduced into the reactor. The reaction conditions were as follows: a ratio of benzene to ethylene of 5, a reaction temperature of 330° C., a reaction pressure of 1.4 MPa, and a weight hourly space velocity of 2.0 h⁻¹.

The reaction results were an ethylene conversion of 99.99% and an ethylbenzene selectivity of 96.21%, after reaction for 500 h.

### Example 7

This example provides a solid acid alkylation catalyst, prepared as follows:
the molecular sieve product of Example 2 was taken and roasted at 540° C. under air atmosphere for 5 h to remove the template agent: it was then subjected to ammonium exchange in 1 mol/L ammonium nitrate solution at 80° C. for 2 h and roasted again at 540° C. for 4 h under air atmosphere to obtain an H-type molecular sieve;
90 g of the H-type molecular sieve was mixed evenly with 15 g of pseudo boehmite, and 55 g of nitric acid solution was added gradually while kneading, and extruded into a φ2.0 mm cylindrical catalyst, which was then truncated into a cylindrical catalyst with a length of 2.5 mm; the catalyst was dried at room temperature for 24 h, and then roasted at 550° C. for 6 h to obtain a finished solid acid alkylation catalyst.

The solid acid alkylation catalyst obtained in this example was applied to catalyze the alkylation reaction of an aromatic hydrocarbon with an olefin as follows:
2 g of the finished solid acid alkylation catalyst was loaded into a fixed-bed reactor and a mixture of benzene and propylene was introduced into the reactor. The reaction conditions were as follows: a ratio of benzene to propylene of 4, a reaction temperature of 150° C., a reaction pressure of 2.5 MPa, and a weight hourly space velocity of 3.0 h⁻¹.

The reaction results were a propylene conversion of 99.97% and an isopropylbenzene selectivity of 99.20%, after reaction for 300 h.

### Example 8

This example provides a solid acid alkylation catalyst, prepared as follows:
the molecular sieve product of Example 3 was taken and roasted at 540° C. under air atmosphere for 5 h to remove the template agent; it was then subjected to ammonium exchange in 1 mol/L ammonium nitrate solution at 80° C. for 2 h and roasted again at 540° C. for 4 h under air atmosphere to obtain an H-type molecular sieve;
90 g of the H-type molecular sieve was mixed evenly with 15 g of pseudo boehmite, and 55 g of nitric acid solution was added gradually while kneading, and extruded into a φ2.0 mm cylindrical catalyst, which was then truncated into a cylindrical catalyst with a length of 2.5 mm. The catalyst was dried at room temperature for 24 h, and then roasted at 550° C. for 6 h to obtain a finished solid acid alkylation catalyst.

The solid acid alkylation catalyst obtained in this example was applied to catalyze the alkylation reaction of an aromatic hydrocarbon with an olefin as follows:
2 g of the finished solid acid alkylation catalyst was loaded into a fixed-bed reactor and a mixture of benzene and n-dodecene was introduced into the reactor. The reaction conditions were as follows: a ratio of benzene to n-dodecene of 4, a reaction temperature of 150° C., a reaction pressure of 3.0 MPa, and a weight hourly space velocity of 2.0 h⁻¹.

The reaction results were a n-dodecene conversion of 99.97% and a 2-alkylbenzene selectivity of 42.01%, after reaction for 300 h.

### Comparative Example 1

This comparative example provides a MWW-structured molecular sieve, prepared as follows:
1.95 g of sodium hydroxide was added to 130 g of deionized water and dissolved with stirring; 2.33 g of sodium metaaluminate was added and dissolved with continuous strong stirring for 1 h; 9.00 g of cyclohexylamine was added slowly with continuous strong stirring for 0.5 h; 80 g of silica sol was added slowly with continuous strong stirring for 3 h; a crystal seed was added in an amount of 5% of the effective SiO₂ mass in the silica sol with continuous strong stirring for 1 h, to produce a crystallized gel; he crystallized gel was crystallized at 140° C. for 72 h; at the end of crystallization, the temperature was reduced to room temperature; the product was washed with deionized water, filtered and dried naturally for 48 h, to obtain the molecular sieve.

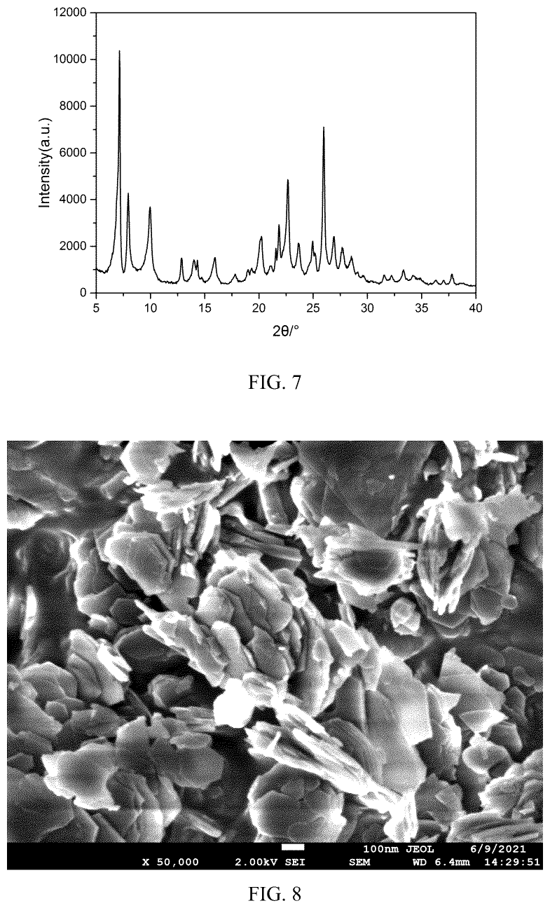

The molecular sieve obtained in this comparative example was characterized by XRD and SEM analysis. The XRD spectrum and SEM image are shown in FIGS. 7 and 8, respectively, which indicate that the molecular sieve has typical characteristic peaks of MWW structure and can be subdivided into MCM-49 molecular sieve. Moreover, the morphology of this molecular sieve product is in the form of ordered stacked nanosheets.

The molecular sieve obtained in this comparative example was characterized by BET analysis after roasting at 540° C. in air atmosphere, and the results of BET test are shown in Table 1.

Based on the test results, it can be seen by comparing Comparative Example 1 with Example 3 that a MWW-structured molecular sieve with high crystallinity and pure phase can be directly synthesized and subdivided into MCM-49 molecular sieve when the crystal seed and cyclohexylamine as a template agent are used. However, cyclohexylamine cannot construct interlayer hydrogen bonding, so such a method is only suitable for the synthesis of MCM-49 molecular sieve, and it is impossible to synthesize other kinds of MWW-structured molecular sieve.

TABLE 1
BET test results of molecular sieves

| | S_{BET}/ | S_{mic}/ | Sₑₓₜ/ | Vₚₒᵣₑ/ | S_{mic}/ |
|---|---|---|---|---|---|
| Sample | m² · g | m² · g | m² · g | cm³ · g | cm³ · g |
| Example 1 | 541.73 | 399.36 | 142.37 | 0.61 | 0.16 |
| Example 2 | 562.27 | 402.33 | 159.94 | 0.66 | 0.17 |
| Example 3 | 504.11 | 353.68 | 150.43 | 0.63 | 0.16 |
| Example 5 | 527.65 | 361.47 | 166.18 | 0.64 | 0.16 |
| Comparative | 419.42 | 362.87 | 56.55 | 0.52 | 0.16 |
| Example 1 | | | | | |

In Example 1, due to the addition of diisopropylamine, the electron and holes from the sp3 hybridization of the nitrogen atoms form hydrogen bonding with the hydrogen in the interlayer Si—OH, and thus the molecular sieve product exhibits properties more similar to those of the MCM-22 (P) molecular sieve, with a lower stack number of basic layers, a reduced thickness of the molecular sieve sheets, an increase in the total surface area and the external specific surface area, and a change in the molecular sieve stacking morphology.

### Comparative Example 2

This comparative example provides a catalyst prepared from the molecular sieve of Comparative Example 1 as follows:
the molecular sieve product of Comparative Example 1 was taken and roasted at 540° C. under air atmosphere for 5 h to remove the template agent; it was then subjected to ammonium exchange in 1 mol/L ammonium nitrate solution at 80° C. for 2 h and roasted again at 540° C. for 4 h under air atmosphere to obtain an H-type molecular sieve;
90 g of the H-type molecular sieve was mixed evenly with 15 g of pseudo boehmite, and 55 g of nitric acid solution was added gradually while kneading, and then extruded into a φ2.0 mm cylindrical catalyst, which was then truncated into a cylindrical catalyst with a length of 2.5 mm; the catalyst was dried at room temperature for 24 h, and then roasted at 550° C. for 6 h to obtain a finished catalyst.

The solid acid alkylation catalyst obtained in this comparative example was applied to catalyze the alkylation reaction of an aromatic hydrocarbon with an olefin as follows:
2 g of the finished catalyst was loaded into a fixed-bed reactor and a mixture of benzene and n-dodecene was introduced into the reactor. The reaction conditions were as follows: a ratio of benzene to n-dodecene of 15, a reaction temperature of 150° C., a reaction pressure of 3.0 MPa, and a weight hourly space velocity of 2.0 h⁻¹. The reaction results were a n-dodecene conversion of 76.32% and a 2-alkylbenzene selectivity of 45.03%, after reaction for 100 h.

It can be seen by comparing Comparative Example 2 with Example 8 that when the MCM-49 molecular sieve is used in the preparation of an alkylation catalyst for the reaction of benzene with n-dodecene, since the thickness of the molecular sieve is greater than that of the MCM-22 molecular sieve, the number of surface catalytically active sites exposed per unit mass of the catalyst decreases, and it is more quickly deactivated by blockage during the reaction and the life of the catalyst is shortened.

### Comparative Example 3

This Comparative Example provides a molecular sieve, prepared by a method as follows:
1.87 g of sodium hydroxide was added to 120 g of deionized water and dissolved with stirring: 2.15 g of sodium metaaluminate was added and dissolved with continuous strong stirring for 1 h; 19.0 g of hexamethyleneimine was added slowly with continuous strong stirring for 0.5 h, to obtain an intermediate solution;
80 g of silica sol was added slowly to the intermediate solution with continuous strong stirring for 3 h, to produce a crystallized gel: the crystallized gel was crystallized at 155° C. for 60 h: at the end of crystallization, the temperature was reduced to room temperature: the product was washed with deionized water, filtered and dried at 120° C. for 4 h, to obtain the molecular sieve product.

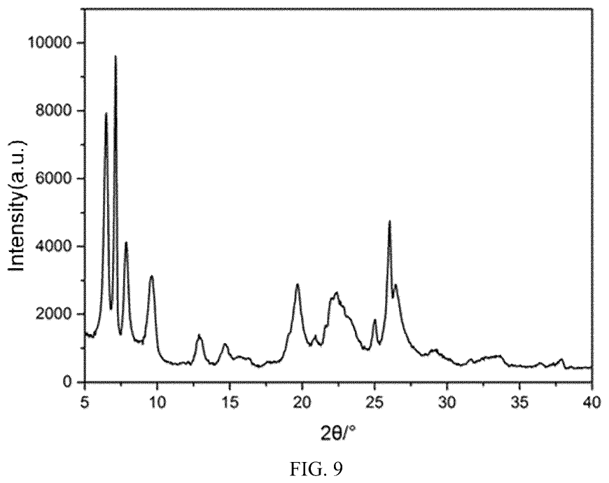

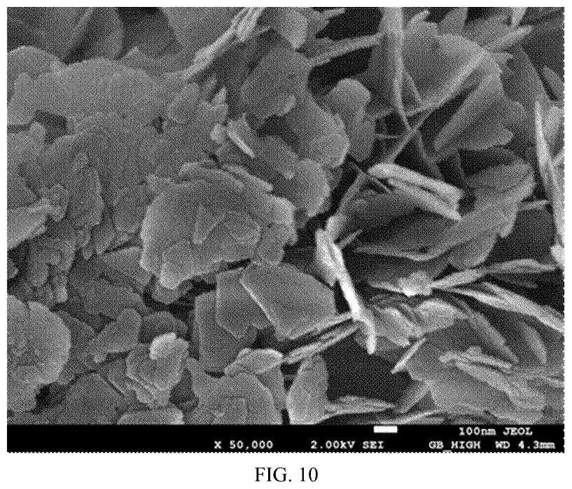

The molecular sieve obtained from this comparative example was characterized by XRD and SEM analyses. The XRD spectrum and SEM image are shown in FIGS. 9 and 10, respectively. The results indicate that the molecular sieve product has typical characteristic peaks of MWW structure and can be subdivided into MCM-49 molecular sieve. Moreover, the molecular sieve morphology is in the form of stacked nanosheets.

The hexamethylenimine used in this comparative example is a typical template agent for the preparation of MCM-22 molecular sieve. Although a MCM-22 molecular sieve with pure phase can be obtained by this method, the template agent hexamethylenimine is used in a large amount, and it is expensive and toxic.

It can be seen by comparing the XRD results of the molecular sieve products of Examples 1 to 5 with the XRD results of Comparative Example 3 that that the molecular sieves of Examples 1 to 5 have a structure similar to that of the MCM-22 molecular sieve, which further confirms that the molecular sieve obtained from SI of the present disclosure has interlayer hydrogen bonding.

1. A method for preparing a solid acid alkylation catalyst, comprising the following steps:
S1: mixing an aluminum source, water, an alkali source, a template agent, a silicon source, and a crystal seed to form a gel, and subjecting the gel to a crystallization reaction to obtain a MWW-structured molecular sieve; wherein the template agent is composed of a primary template agent and a secondary template agent in a molar ratio of 0.5-20:1;
wherein the primary template agent is cyclohexylamine, and the secondary template agent comprises at least one of diisopropylamine, di-n-butylamine, diisobutylamine, 1,4-diazabicyclo[2.2.2]octane, 1,6-hexanediamine and N,N,N,N-tetramethyl-1,6-hexanediamine;
S2: roasting the MWW-structured molecular sieve obtained from S1 to remove the template agent, followed by ammonium exchange and roasting again, to obtain an H-type molecular sieve; and
S3: mixing the H-type molecular sieve obtained from S2 with an inorganic oxide and an inorganic acid solution, kneading and shaping, drying and then roasting, to obtain the solid acid alkylation catalyst.

2. The method for preparing a solid acid alkylation catalyst according to claim 1, wherein in the gel in S1, the molar ratio of the silicon source in terms of SiO₂, the aluminum source in terms of Al₂O₃, the alkali source in terms of alkali metal oxide and the template agent is 1:0.005-0.05:0.03-0.50:0.1-0.75; and
the mass ratio of the crystal seed on dry mass basis to the silicon source in terms of SiO₂ is 0.01-0.25:1.

3. The method for preparing a solid acid alkylation catalyst according to claim 1, wherein in S1, the crystal seed is a molecular sieve having a MWW topology.

4. The method for preparing a solid acid alkylation catalyst according to claim 1, wherein the crystal seed comprises a MCM-22 molecular sieve or a MCM-49 molecular sieve without template agent removal.

5. The method for preparing a solid acid alkylation catalyst according to claim 1, wherein in S1, the silicon source comprises at least one of silica sol, solid silica gel, white carbon black and water glass.

6. The method for preparing a solid acid alkylation catalyst according to claim 1, wherein in S1, the alkali source comprises sodium hydroxide and/or potassium hydroxide.

7. The method for preparing a solid acid alkylation catalyst according to claim 1, wherein in S1, the aluminum source comprises at least one of sodium metaaluminate, aluminum sulfate, alumina and pseudo boehmite.

8. The method for preparing a solid acid alkylation catalyst according to claim 1, wherein in S1, the crystallization reaction temperature is 120-170° C., and the reaction time is 12-120 h.

9. The method for preparing a solid acid alkylation catalyst according to claim 1, wherein in S2, the roasting is each carried out under an air atmosphere, the roasting temperature is each independently 490-590° C., and the roasting time is each independently 0.5-72 h.

10. The method for preparing a solid acid alkylation catalyst according to claim 1, wherein in S2, the ammonium exchange is carried out in an ammonium nitrate solution with a concentration of 0.1-10 mol/L; the ammonium exchange temperature is 30-120° C., and the ammonium exchange time is 0.5-72 h.

11. The method for preparing a solid acid alkylation catalyst according to claim 1, wherein in S3, the mass ratio of the H-type molecular sieve to the inorganic oxide is 10-95:5-90.

12. The method for preparing a solid acid alkylation catalyst according to claim 1, wherein in S3, the inorganic oxide comprises at least one of aluminium oxide, pseudo boehmite, boehmite and aluminum hydroxide;
the inorganic acid solution comprises at least one of nitric acid solution, phosphoric acid solution, hydrochloric acid and sulfuric acid solution.

13. The method for preparing a solid acid alkylation catalyst according to claim 1, wherein in S3, the roasting is carried out under an air atmosphere, the roasting temperature is 490-590° C., and the roasting time is 0.5-72 h.

14. A solid acid alkylation catalyst obtained from the method for preparing a solid acid alkylation catalyst according to claim 1.

15. Use of the solid acid alkylation catalyst obtained from the method for preparing a solid acid alkylation catalyst according to claim 1 in the alkylation reaction of an aromatic hydrocarbon with an olefin.

16. The use according to claim 15, wherein the aromatic hydrocarbon is selected from at least one of benzene, alkylbenzene, naphthalene, alkylnaphthalene, anthracene and alkylanthracene.

17. The use according to claim 15, wherein the olefin is selected from olefins having 2 to 20 carbon atoms.

18. The use according to claim 15, wherein the alkylation reaction conditions include a reaction temperature of 100-385° C., a reaction pressure of 0.5-7 MPa, and a weight hourly space velocity of 0.1-12 h⁻¹.

19. The use according to claim 18, wherein the alkylation reaction conditions include a reaction temperature of 120-350° C., a reaction pressure of 0.5-5 MPa, and a weight hourly space velocity of 0.5-10 h⁻¹.

20. The use according to claim 15, wherein the feeding molar ratio of the aromatic hydrocarbon to the olefin is 0.05-200.
